(19) United States
(12) Patent Application Publication
(10) Pub. No.: US 2011/0237638 A1
(43) Pub. Date: Sep. 29, 2011

(54) METRONIDAZOLE-BASED DERMATOLOGICAL FOAM AND EMULSIONS FOR THE PREPARATION THEREOF

(75) Inventors: Dov TAMARKIN, Maccabim (IL); Doron Friedman, Karmei-Yosef (IL)

(73) Assignee: GALDERMA S.A., Cham (CH)

(21) Appl. No.: 13/154,478

(22) Filed: Jun. 7, 2011

Related U.S. Application Data
(63) Continuation of application No. 12/076,114, filed on Mar. 13, 2008, now abandoned.
Application No. 12/076,114 is a continuation of application No. PCT/FR2006/002038, filed on Sep. 5, 2006.

(30) Foreign Application Priority Data
Sep. 13, 2005 (FR) 0509340

(51) Int. Cl. A61K 31/4164; A61P 17/00; B67D 7/06; B65D 83/14

(52) U.S. Cl. 514/398; 222/1; 222/635

(57) ABSTRACT

Oil-in-water emulsions expandable into stable foams having diminished inhibitor of skin breathing and useful for the treatment of dermatological conditions/afflictions, e.g., rosacea, advantageously contain:
(a) 72% to 76% of water;
(b) 0.1% to 5% of a gelling agent for the aqueous phase of the emulsion;
(c) 0.75% to 2% of metronidazole;
(d) 5% to 10% of mineral oil;
(e) 0.5% to 10% of a surfactant;
(f) 0.5% to 5% of a fatty acid;
(g) 0% to 4% of a metronidazole absorption promoter (c); and
(h) 0.1% to 5% of a preservative selected from the group consisting of methylparaben, propylparaben, phenoxyethanol and mixtures thereof.

### CROSS-REFERENCE TO COMPANION APPLICATION

Copending U.S. patent application Ser. No. ______ [Attorney Docket No. 1034227-0001187], filed concurrently herewith, hereby expressly incorporated by reference and also assigned to the assignee hereof.

### CROSS-REFERENCE TO PRIORITY/PCT APPLICATIONS

This application is a continuation of U.S. application Ser. No. 12/076,114, filed Mar. 13, 2008, and claims priority under 35 U.S.C. §119 of FR 0509340, filed Sep. 13, 2005, and is a continuation of PCT/FR 2006/002038, filed Sep. 5, 2006 and designating the United States (published in the French language on Mar. 22, 2007 as WO 2007/031621 A2; the title and abstract were also published in English), each hereby expressly incorporated by reference in its entirety and each assigned to the assignee hereof.

### BACKGROUND OF THE INVENTION

1. Technical Field of the Invention

The present invention relates to metronidazole-based foam compositions, in particular as topical dermatological compositions, especially for the treatment of dermatoses, such as rosacea.

2. Description of Background and/or Related and/or Prior Art

Metronidazole, or 1-(2-hydroxyethyl)-2-methyl-5-nitroimidazole, is the compound having the following formula (I):

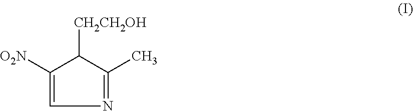

This compound and protocol for the preparation thereof are well known and have been described, in particular, in U.S. Pat. No. 2,944,061.

Metronidazole is an acknowledged anti-bacterial and anti-parasitic active agent useful for the treatment of many conditions/afflictions. This compound is known in particular as being particularly effective in the treatment of skin disorders such as rosacea.

Rosacea is a chronic skin condition which affects mainly adults. It is a type of dermatosis with recurrent symptoms, including in particular erythemas, papules, pustules, rhinophymas and/or telangiectasias, which manifests itself mainly in the region of the nose, the cheeks and the forehead.

For the treatment of such conditions, metronidazole is preferably administered by the topical route. Indeed, administration by the systemic route, in particular by the oral route, leads, in most cases, to undesirable side effects, such as gastrointestinal intolerance or vaginitis, to which other chronic disorders may also be added in the case of a long-term administration.

Various topical formulations have been proposed for the topical administration of metronidazole, which are mainly oil-based creams or oily ointments (in particular compositions based on petroleum jelly). These oily compositions have in general the advantage of being able to contain large quantities of metronidazole, in a state available for topical application, but they are in practice poorly suited to dermatological use.

Indeed, in addition to their greasy feel, which is unpleasant for the patient, they most often require the presence of ingredients which are found to exhibit comedogenic, acnegenic, drying and/or irritating properties for the skin in the shorter or longer term. In many cases, patients treated with compositions of this type additionally feel sensations of burning or urtication. Another disadvantage of these compositions is that they have a tendency to disrupt or even inhibit the phenomena of skin breathing when they are applied to the skin.

To replace this type of oily compositions, it has been proposed, in U.S. Pat. No. 4,837,378, to administer metronidazole in the form of an aqueous gel. Such a gel has, among other advantages, that of not having the greasy feel of the abovementioned oily compositions, and especially that of not involving the use of comedogenic, acnegenic, drying or irritating agents necessary in the oily compositions.

Nevertheless, despite these advantages, it has been found that the topical application of a gel such as those described in U.S. Pat. No. 4,837,378 to the skin most often promotes a disruption or an inhibition of the phenomena of skin breathing, similar to those observed with the abovementioned oily compositions. In any event, the application of such a gel to the skin is in general not favorable to normal breathing of the skin.

### SUMMARY OF THE INVENTION

The present invention provides novel compositions which are particularly well suited for topical administration of metronidazole to the skin. In this context, the present invention preferably provides compositions which present the advantages of the compositions of U.S. Pat. No. 4,837,378 while promoting a lower inhibition of the phenomenon of skin breathing.

To this effect, the present invention provides compositions comprising metronidazole, having the form of a foam, obtained from an emulsion subjected to the effect of a gas.

The preparation of such a foam from an emulsion is a known technique, as well as the use of the foam obtained for the topical administration of active ingredients, in particular to the skin or the mucous membranes. Reference may thus be made, in particular, to U.S. Pat. Nos. 6,126,920 and 6,423,323, and WO 2004/037225.

The foams of the abovementioned type are in general obtained by placing an emulsion of a suitable formulation into an aerosol container with a gas under pressure. When the composition is released at atmospheric pressure (for example through an outlet nozzle communicating with the emulsion) the extremely high pressure which exists in the container pushes a mixture of emulsion and gas under pressure out of the container. The expansion of the gas present in this mixture then leads to the formation of a “foam”, namely, a structure formed of agglomerated bubbles filled with the gas and whose walls are formed by the emulsion (this process is similar to that observed more commonly with the systems for delivering shaving foams). The foams obtained in this context are in general not stable long term, and they are most often applied just after their formation at the outlet of the aerosol container.

It is known that foams constitute vehicles which are particularly suitable for delivering active ingredients, which allow in particular an improvement in the absorption by the skin or the mucous membranes compared with the more customary compositions of the gel, cream or ointment type. However, to obtain such an effect, the nature of the emulsion employed is in general to be adapted according to the nature of the active ingredient. Indeed, according to its chemical nature, an active ingredient can induce an excessively rapid destabilization of the foam, or even an inhibition of its formation, not permitting the desired application. In this respect, it is most often found to be necessary to adapt on a case-by-case basis the formulation of the emulsion to the active ingredient to be delivered in order to obtain a foam of the required quality.

The present invention provides emulsions of a very specific formulation, which allow the preparation of foams which are particularly suited to a topical delivery of metronidazole, which is useful, in particular, for the treatment of skin conditions such as rosacea. This invention provides in particular emulsions having a sufficiently low viscosity to allow the delivery of the foam from an aerosol container.

The present invention also provides emulsions suitable for preservation and storage in a closed chamber in the presence of a gas under pressure, preserving over time the stability of the structure of the emulsion and its foaming properties and the integrity of the metronidazole.

### DETAILED DESCRIPTION OF BEST MODE AND SPECIFIC/PREFERRED EMBODIMENTS OF THE INVENTION

More particularly in a first embodiment, the present invention features metronidazole-based oil-in-water emulsions, in particular expandable into the form of a foam under the effect of a gas, comprising, by mass relative to the total mass of the emulsion:

(a) 72% to 76% of water;

(b) 0.1% to 5% of a gelling agent for the aqueous phase of the emulsion;

(c) 0.75% to 2% of metronidazole;

(d) 5% to 10% of mineral oil;

(e) 0.5% to 10% of a surfactant;

(f) 0.5% to 5% of a fatty acid;

(g) 0% to 4% of a metronidazole absorption promoter; and

(h) 0.1% to 5% of a preservative selected from the group consisting of methylparaben, propylparaben, phenoxyethanol and mixtures thereof.

The specific formulation of the emulsions of the invention makes it possible to obtain foams which possess a texture and a stability which are particularly well suited to a pleasant, easy and effective application of metronidazole onto the skin.

The specific emulsions of the present invention provide foams of firm, creamy and light consistency, which have in general a very fine bubble structure, which makes them particularly pleasant to apply. It should moreover be emphasized that the foams obtained generally do not exhibit a greasy feel despite the fact that they comprise compounds of an oily nature (in particular mineral oil).

Furthermore, the structure of the foams obtained from the emulsions of the present invention has a very particular stability: this stability is sufficiently high to allow good handling and easy application of the foam, but the foam nevertheless becomes destabilized under the effect of a light massage during its spreading, which makes it possible to very easily bring about effective penetration of metronidazole in the area treated. Thus, the foams obtained according to the present invention may be applied both to very localized areas of the skin and to larger areas, and they allow distribution and uniform absorption of metronidazole in the treated areas without having to massage the treated area intensively in order to effect penetration of the composition, which makes it possible in particular to avoid irritations in the skin areas where the metronidazole is applied.

Thus, the emulsions according to the invention provide foams which have good stability and good cosmetic acceptability, which are well tolerated, which maintain the integrity of the active ingredient and which improve its release-penetration capacities.

The emulsions of the invention contain, in general, at least 0.75% by mass of metronidazole relative to the total mass of the emulsion, preferably at least 1%. These quantities thus provide, in the presence of propellant gas, foams preferably containing 0.75% or 1% of metronidazole.

The water content of the emulsions according to the invention is for its part in general less than or equal to 76% by mass, and it is preferably from 72% to 76% by mass, relative to the total mass of the emulsion.

Preferably, the metronidazole is mainly present in the dissolved state in the aqueous phase of the emulsion. To this effect, the presence of the gelling agent (b) often plays an important role.

The gelling agent (b) present in the emulsion has the role of increasing the viscosity of the aqueous phase of the emulsion, which makes it possible in particular to improve the stabilization of this phase and its binding character, which leads to a good homogeneity of the distribution of metronidazole in the composition and to foams having the desired texture and stability being obtained. This gelling agent (b) may be selected in particular from among:

natural polymers such as xanthan gum, carrageenan gum, guar gum, carob gum, gum tragacanth, quince seed extract; alginates such as sodium alginate; sodium caseinate; albumin; agar-type gelatin; and starch;

semisynthetic polymers such as cellulose ethers (in particular hydroxyethylcellulose, methylcellulose, carboxymethylcellulose, hydroxypropylmethylcellulose), polyvinyl alcohols, hydroxypropylated guar gum, modified starches such as soluble starches, cationic celluloses, cationic guar gums;

synthetic polymers such as carboxyvinyl polymers, polyvinylpyrrolidone, polyvinyl alcohol, polymers of polyacrylic and/or polymethacrylic acids, polyvinyl acetates, polyvinyl chlorides, polyvinylidenes, or carboxyvinyl polymers such as those marketed by Goodrich under the trademark Carbopol resin (especially Carbopol 934, Carbopol 940, Carbopol 950, Carbopol 980, Carbopol 951 and Carbopol 981); and

mixtures of these compounds.

According to a particular embodiment, the gelling agent (b) comprises a mixture of a natural thickening gum and a cellulose ester, for example a mixture of xanthan gum and methylcellulose. A particularly advantageous gelling system of this type comprises from 0.2% to 0.5% by mass (preferably on the order of 0.3% to 0.4% by mass) of xanthan gum, and from 0.2% to 0.5% by mass (preferably on the order of 0.3% to 0.4% by mass) of methylcellulose, relative to the total mass of the emulsion.

Regardless of its nature, the gelling agent (b) is most often present in an amount of from 0.3% to 2% by mass relative to the total mass of the emulsion, and an amount greater than 1% by mass is in general not required to obtain the desired increase in viscosity. Preferably, the gelling agent (b) is present in a quantity of from 0.4% to 1% by mass, for example from 0.5% to 0.9% by mass, preferably from 0.6% to 0.8% by mass, relative to the total mass of the emulsion.

The oily phase of the emulsions of the invention is based on a mineral oil (d). The expression “mineral oil” means a mixture of aliphatic, naphthalene and aromatic hydrocarbons which is liquid at room temperature (namely at least from 10° C. to 35° C., this oil remaining in general liquid, without substantial formation of crystals at temperatures greater than 0° C.). It is preferably a petroleum derivative, advantageously such as those listed under the CAS registry number (Chemical Abstract Service Registry Number) 8012-95-1. The viscosity of the mineral oil used is preferably from 10 to 100 mPas (mPascal·second), more preferably from 15 to 40 mPas, at 25° C.

This mineral oil (d) may be present in the emulsion in a relatively large quantity, without leading to a greasy feel in the foam finally obtained. According to an advantageous embodiment, the emulsion has a mineral oil (d) content greater than or equal to 6% by mass, this content being preferably from 6% to 8% by mass, for example from 6.5% to 7.5% by mass, and more preferably on the order of 7% to 7.2% by mass, relative to the total mass of the emulsion.

The mineral oil (d) plays, inter alia, a role of emollient in the form ultimately obtained, namely, it improves the lipid content of the skin by providing an emollient effect.

In addition to the mineral oil (d), the emulsions of the invention may comprise other agents which present such an emollient effect. For this effect, the emulsion advantageously comprises at least one ester (i) as emollient agent. Where appropriate, this ester is advantageously present in the emulsion in an amount of 1% to 10% by weight (for example from 4% to 8%) relative to the total mass of the emulsion. This ester is preferably selected from among isopropyl and diisopropyl esters, such as isopropyl myristate, isopropyl palmitate, diisopropyl dimerate, diisopropyl adipate, isopropyl isostearate or isopropyl lanolate; glycerides (glyceryl esters), and more particularly triglycerides; isostearic acid esters; dimethyl isosorbate, cetyl lactate, cetyl ricinoleate, tocopheryl acetate, tocopheryl linoleate, cetyl acetate, pentaerythrityl tetrastearate, neopentylglycol dicaprylate and/or dicaprate, isononyl isononanoate, isotridecyl isononanoate, myristyl myristate, triisocetyl citrate, octyl dodecanoate and octyl hydrostearate; and mixtures of these esters, for example in the form of vegetable or animal oils (soyabean or lanolin oils for example). Isopropyl myristate and the triglycerides are particularly preferred in this context. Among the triglycerides, the triglycerides of C8 to C10 fatty acids, such as, for example, those known under the name MYGLIOL, will advantageously be used.

The surfactant (e) of the emulsions of the invention may be selected, as a general rule, from among most of the compounds capable of bringing about the emulsification of an oily phase in an aqueous phase. Thus, the surfactant (e) may for example be selected from among the anionic, cationic, nonionic, zwitterionic and amphoteric emulsifiers, and ampholites customarily used in dermatological compositions. Examples of such compounds include, in particular, polyoxyethylenated sorbitan esters (polysorbate), polyoxyethylenated fatty acid esters such as Myrj 45, Myrj 49 and Myrj 59; the polyoxyethylenated alkyl ethers such as polyoxyethylenated cetyl ether, polyoxyethylenated palmityl ether, polyoxyethylenated hexadecyl oxide ether, polyethylenated cetylglycol ether, brij 38, brij 52, brij 56 and bryj W1; sucrose esters, partial sorbitan esters such as sorbitan monolaurate, mono- or diglycerides, isoceteth-20, sodium methylcocoyl taurate, sodium methyloleyl taurate, sodium lauryl sulfate, lauryl sulfate and betaines. Preferably, the surfactants (e) used have an HLB (Lipophilic/Hydrophilic Balance) of at least 9, preferably greater than 9.

To obtain the optimum properties for the foam, it is preferable to use surfactants selected from among polyoxyethylenated sorbitan esters such as polysorbate 80 (polyoxyethylene (20) sorbitan monooleate, or Tween 80) or polyoxyethylene (20) sorbitan monostearate (Tween 60); polyethylenated glycol esters such as, for example, PEG-40 stearate, and monoglycerides such as, for example, glyceryl monostearate.

Regardless of its nature, the surfactant (e) is preferably present in the emulsion in an amount of from 3% to 8%, preferably from 3% to 7%, for example from 4% to 6% by mass relative to the total mass.

According to an advantageous embodiment, the surfactant (e) is a mixture of several surfactants. In this context, it is possible to advantageously use, as surfactant (e), a mixture of polyethylene glycol ester, polyoxyethylenated sorbitan ester and monoglyceride (glyceryl monoester). Typically, the surfactant (e) comprises a mixture of:

from 2% to 5%, typically on the order of 3% to 4% of the polyethylene glycol ester; and

from 0.8% to 1.2% of a polyoxyethylenated sorbitan ester; and

from 0.4% to 1% of a monoglyceride.

The role of the surfactant(s) (e) present in the emulsions of the invention is double. On the one hand, all or some of these agents provide an emulsifying effect, which allows the formation and subsequent stabilization of the emulsion. On the other hand, the surfactants present in the emulsion bring about a surface modifying effect at the interfaces of the liquid/gas type, which makes it possible to bring about the formation of the foam from the emulsion.

The particular stability and texture of the metronidazole-based foams of the invention are also due to the specific presence of the fatty acid (f) which plays a role of consistency agent and which makes it possible, in combination with the surfactants, to bring about sufficient stabilization of the foam to allow its appropriate application and to confer its firm and creamy consistency on the foam. Without wishing to be bound by any particular theory, the fatty acid may play in particular a role of promoter for the surfactants, enhancing the emulsification capacities and the foaming properties of the composition.

The fatty acid (f) included in the compositions of the present invention as consistency agent advantageously contains at least one site of unsaturation. This acid is preferably selected from among fatty acids having at least 16 carbon atoms, such as hexadecanoic acid (C₁₆), stearic acid (C₁₈), arachidic acid (C₂₀), behenic acid (C₂₂), octacosanoic acid (C₂₈), and mixtures of these compounds. Stearic acid is most particularly preferred. According to an advantageous embodiment, the fatty acid (f) is stearic acid.

Regardless of its nature, the fatty acid (f) is advantageously present in the composition in an amount of from 1% to 2% by mass, preferably from 1% to 1.5% by mass, and preferably from 1.1% to 1.2% by mass, relative to the total mass of the emulsion.

According to one advantageous embodiment, the emulsion additionally contains a metronidazole (c) absorption promoter (g).

The expression “absorption promoter” means an agent which enhances the absorption of metronidazole into the skin, in particular by increasing the rate of diffusion of the active ingredient through the tissues.

This absorption promoter may be, for example, a polyol, in particular propylene glycol, exylene glycol or diethylene glycol, ethylene glycol and glycerol; terpenes, diterpenes or triterpenes, in particular limonene; terpenol, for example 1-menthol; dioxolane; sulfoxides such as dimethyl sulfoxide DMSO, dimethylformamide, methyl and dodecyl sulfoxide, dimethylacetamic.

Propylene glycol is most particularly preferred as the absorption promoter (g).

In addition to the abovementioned compounds, the emulsion according to the invention comprises a preservative (h), preferably in an effective quantity to inhibit microbial growth in the emulsion during storage thereof. Preferably, this compound is present in an amount of from 0.1% to 5% by mass relative to the total mass of the emulsion.

Advantageously, it is a preservative of the paraben family in combination with phenoxyethanol. More preferably, this preservative is a mixture of methylparaben and propylparaben with phenoxyethanol, in any proportions. Preferably, this preservative is a mixture of:

12 to 15% of methylparaben,

4 to 8% of propylparaben, and

78 to 84% of phenoxyethanol,

the percentages being expressed by weight relative to the total weight of the preservative.

According to another particular embodiment, the emulsion may in fact comprise ethylenediaminetetraacetic acid (EDTA) or one of its salts as additional ingredient.

EDTA, commonly employed in dermatological compositions, is useful in particular for chelating metal cations which may be present as impurities in the composition, which makes it possible in particular to avoid undesirable side effects in some patients. Where appropriate, the emulsion advantageously contains EDTA, preferably in an amount of 0.01% to 0.1% by mass, and typically in an amount on the order of 0.5% by mass relative to the total mass of the emulsion.

According to one particularly advantageous embodiment, the emulsion of the present invention is an emulsion (termed hereinafter emulsion E⁰), which comprises, by mass relative to the total mass of the emulsion:

(a) 72% to 76% of water;

(b) a gelling system for the aqueous phase of the emulsion, comprising:
0.2% to 0.5%, preferably 0.3% to 0.4%, of xanthan gum; and
0.2% to 0.5%, preferably 0.3% to 0.4%, of methylcellulose;

(c) 0.75% to 2% of metronidazole;

(d) 6% to 8%, preferably 6.5% to 7.5%, of mineral oil; and

(e) a surfactant comprising a mixture of:
2% to 5%, preferably on the order of 3% to 4%, of a polyethylene glycol ester; and
0.8% to 2%, preferably on the order of 1% to 1.5%, of a polyoxyethylenated sorbitan ester; and
0.4% to 1%, preferably on the order of 0.5% to 0.8%, of a monoglyceride;

(f) 1% to 1.5%, preferably from 1.1% to 1.2%, of stearic acid;

(g) 0% to 4% of propylene glycol (most often, the composition does not comprise propylene glycol, or it contains on the order of 3% to 4% thereof;

(h) 0.1% to 2% of a preservative selected from the group consisting of methylparaben, propylparaben, phenoxyethanol and mixtures thereof; and

(i) 0.1% to 10%, preferably from 5% to 9%, of an emollient ester, preferably selected from among isopropyl myristate and fatty acid triglycerides, in particular C8-C10 fatty acid triglycerides.

According to a first particularly advantageous embodiment, this emulsion E⁰ comprises, by mass relative to the total mass of the emulsion:

(a) 72% to 76% of water;

(b) a gelling system for the aqueous phase of the emulsion, comprising:
from 0.3% to 0.4% of xanthan gum; and
from 0.3% to 0.4% of methylcellulose;

(c) 0.75% to 2% of metronidazole;

(d) 6.5% to 8.5%, preferably 7% to 7.2%, of mineral oil;

(e) a surfactant comprising a mixture of:
3 to 4% of PEG-40 stearate;
1% to 1.5% of polysorbate 80 and
0.5% to 0.8% of a glyceryl monostearate;

(f) from 1% to 1.5%, preferably from 1.1% to 1.2%, of stearic acid;

(g) 0% to 4% of propylene glycol (according to a particular variant, the emulsion E⁰ according to this embodiment does not contain propylene glycol);

(h) 0.1% to 2% of a preservative selected from the group consisting of methylparaben, propylparaben, phenoxyethanol and mixtures thereof, for example from 1.2 to 1.7% of a mixture of methylparaben, propylparaben and phenoxyethanol; and

(i) 5% to 9%, preferably 6% to 8%, of isopropyl myristate or fatty acid triglycerides, as emollient agent.

According to another advantageous embodiment, the emulsion E⁰ comprises, by mass relative to the total mass of the emulsion:

(a) 72% to 76% of water;

(b) a gelling system for the aqueous phase of the emulsion, comprising:
from 0.3% to 0.4% of xanthan gum; and
from 0.3% to 0.4% of methylcellulose;

(c) from 0.75% to 2% of metronidazole; (d) from 6.5% to 7.5%, preferably from 7% to 7.2%, of mineral oil, and

(e) a surfactant comprising a mixture of:
3% to 4% of PEG-40 stearate;
1% to 1.5% of polysorbate 80, and
0.5% to 0.8% of glyceryl monostearate;

(f) from 1% to 1.5%, preferably from 1.1% to 1.2%, of stearic acid;

(g) from 2% to 4%, preferably from 3% to 4%, of propylene glycol;

(h) 0.1% to 2% of a preservative selected from the group consisting of methylparaben, propylparaben, phenoxyethanol and mixtures thereof, preferably from 1.2 to 1.7% of a mixture of methylparaben, propylparaben and phenoxyethanol; and

(i) 5% to 9%, preferably 6 to 8%, of isopropyl myristate or fatty acid triglycerides, as emollient agent.

According to another particular embodiment, the present invention features a method for preparing the abovementioned emulsions. In general, this method comprises the following successive steps:

a preferably monophasic aqueous medium A is prepared which comprises water (a), the gelling agent (b), the metronidazole (c), the surfactant (e), the preservative (h) of the paraben type, and where appropriate the absorption promoter (g), preferably a glycol,

a preferably monophasic hydrophobic medium H is prepared which comprises the mineral oil (d) and any other constituent of the fatty phase, such as the stearic acid (f),

the media A and H thus obtained are mixed, the system thus formed is emulsified in the presence of the surfactant (e) and the stearic acid (f);

the preservative of the phenoxyethanol type is added at the end of the emulsification.

Advantageously, this method is performed by preparing the phase A by heating the water (a) to a temperature of from 40° C. to 80° C., preferably to 70° C., in which the gelling agent (b) is dispersed. As a general rule, it is found to be advantageous to incorporate the surfactant (e) into the phase A before mixing the two phases. Moreover, if the emulsion contains water-soluble ingredients, for example a glycol such as propylene glycol, it is also preferable to incorporate them into the phase A before mixing the two phases A and H. Also added at this stage are the preservatives of the paraben type. The metronidazole (c) is then incorporated into the phase A.

The phase H is prepared by melting on a water bath, after weighing, all the lipophilic constituents of the emulsion. In particular, it is advantageous that the phase H comprises the fatty acid (f) of the stearic acid type and, where appropriate, the ester (i) of the isopropyl myristate type used as emollient agent. The whole is heated to a temperature of from 40° C. to 80° C., preferably to 70° C., with magnetic stirring.

The emulsification is carried out at a temperature from 40° C. to 80° C., preferably at 70° C., by gently introducing the fatty phase into the aqueous phase, with Rayneri stirring (from 500 to 800 rpm, preferably 640 rpm). The temperature and the stirring are maintained for a period of from 5 min to 20 min, preferably 10 min.

The emulsion is then allowed to cool to a temperature of less than 50° C. with slower stirring (200 to 480 rpm, preferably 400 rpm). The preservative of the phenoxyethanol type is introduced at this stage after emulsification. The stirring is then stopped and the emulsion is allowed to cool to room temperature. The remainder as water (qs) is then added. The homogeneity of the emulsion is then monitored under a microscope.

Thus, the emulsion E⁰ may typically be obtained according to a method which comprises the following successive steps:

a preferably monophasic aqueous medium A is prepared which comprises the water (a), the gelling agent (b), the metronidazole (c), the surfactant (e), the absorption promoter (g) of the propylene glycol type and the preservative (h) of the paraben type.

In this context, the metronidazole (c) is advantageously introduced into the aqueous medium containing the propylene glycol (g), for example in an aqueous solution comprising the gelling agent (b), the surfactant (e), the propylene glycol (g) and the preservative of the paraben type (h).

a preferably monophasic hydrophobic medium H is prepared which comprises the mineral oil (d), the stearic acid (f) and the ester (i); and

the media A and H thus obtained are mixed, and the system thus formed is emulsified;

the preservative of the phenoxyethanol type is added at the end of the emulsification.

According to yet another specific embodiment, this invention features a method for preparing a composition in the form of a metronidazole-based foam, by mixing an emulsion of the above-mentioned type with a gas.

Most typically, the mixture is obtained by introducing the emulsion into an aerosol container with a propellant gas under pressure, and then releasing the formulation thus obtained, the foam is thereby obtained at the outlet of the aerosol container. The foam may then be obtained just at the time of its application.

The aerosol container employed in this embodiment is preferably a container of the shaving foam can type, namely, a closed container under pressure, comprising an outlet nozzle communicating with the emulsion and containing the gas under pressure.

According to a particular embodiment, the aerosol containers for delivering a foam according to the abovementioned method comprise:

an emulsion of the abovementioned type; and

a propellant gas under pressure constitute another specific subject of the present invention.

The “propellant gas” according to the present invention is a compound or a mixture of compounds which are gaseous at the temperature and atmospheric pressure for using the foam. This propellant gas may however be present both in the gaseous state and in the liquid state in the aerosol container into which it is introduced. It is advantageously a gaseous hydrocarbon at ambient temperature and atmospheric pressure, such as butane, propane, isobutane and one of the mixtures thereof, such as the mixture of butane and propane, for example. The propellant gas is used according to the present invention in proportions ranging from 10% to 20%, preferably 14% by weight of the composition.

The present invention also features compositions in the form of a metronidazole-based foam which can be prepared according thereto.

The compositions in the form of foams which are obtained from the abovementioned emulsions also constitute another embodiment of the present invention.

These compositions, in the form of foams, are in particular suitable for the prophylactic or therapeutic treatment of skin conditions via topical administration, in particular in human beings, and whether regime or regimen. Such skin conditions are rosacea, or various forms of acne, such as acne vulgaris, steroid acne, acne conglobata or nodulocystic acne, or certain types of dermatitis, such as perioral or seborrhoeic dermatitis.

These different uses of the foams constitute another embodiment of the present invention. Thus, the present invention also features the use of an emulsion indicated above for the preparation of a dermatological foam useful for the prophylactic or therapeutic treatment of a skin condition, in particular rosacea, by the topical route.

The foam compositions according to the invention permit good release-penetration of the active agent through the skin as indicated in the following examples.

The compositions according to the invention have in particular been the subject of a study for the optimization of the preservative system as described in the examples which follow.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Preparation of a Formulation F1 for Delivering a Foam Based on Metronidazole and Isopropyl Myristate

A metronidazole-based emulsion E1 according to the invention was prepared according to the protocol below. The quantities of the various compounds used are presented in Table I below.

Preparation of the Aqueous Phase (A1):

Xanthan gum and methylcellulose (gelling agents), PEG-40 stearate, polysorbate 80 and glyceryl monostearate (surfactants) were introduced into stirred purified water heated to 70° C. The medium obtained was then adjusted to 60° C. While maintaining the stirring, a mixture of methylparaben and propylparaben (a portion of the preservatives) was introduced, followed by metronidazole.

A metronidazole-based aqueous phase A1 was thus obtained.

Preparation of the Hydrophobic Phase (H1):

Stearic acid and mineral oil were introduced into a beaker. The mixture is then melted on a water bath and then homogenized and heated to 70° C. while the stirring is maintained. Isopropyl myristate (emollient agent) was incorporated into the medium thus obtained, maintained under stirring.

A hydrophobic phase H1 was thus obtained.

Synthesis of the Emulsion (E1):

The phase H1, heated to 70° C., was gradually introduced into the phase A1 maintained at 70° C. and maintained under stirring. The medium thus obtained was then subjected to homogenization with Ultraturax.

The emulsion is allowed to cool to a temperature of less than 50° C., with slow stirring. The preservative phenoxyethanol is then added at the end of the emulsification. The stirring is maintained and the emulsion is allowed to cool to room temperature.

An emulsion E1 of the oil-in-water type was thus obtained which has the composition given in Table I below, where the percentages indicated are expressed by mass relative to the total mass of the emulsion.

TABLE I
Composition of the Emulsion E1:

| Constituent | Content (% by mass) |
|---|---|
| Purified water | 75.83 |
| Xanthan gum | 0.36 |
| Methylcellulose | 0.36 |
| PEG-40 stearate | 3.57 |
| Polysorbate 80 | 1.19 |
| Glyceryl monostearate | 0.60 |
| Methylparaben | 0.18 |
| Propylparaben | 0.06 |
| Phenoxyethanol | 1.19 |
| Metronidazole | 1.19 |
| Mineral oil | 7.14 |
| Stearic acid | 1.19 |
| Isopropyl myristate | 7.14 |

Packaging:

The emulsion E1 was introduced into an aerosol container. After closing the container, a butane and propane mixture was introduced under pressure as propellant gas. The formulation F1 obtained in the aerosol container has the following composition:

Emulsion E1: 84% by mass

Propellant gas: 16% by mass

In other words, the formulation contained in the container has the composition indicated in the following table II, where the percentages indicated are expressed by mass relative to the total mass of the formulation.

TABLE II
Composition of the Formulation F1 in the aerosol container:

| Constituent | Content (% by mass) |
|---|---|
| Purified water | 63.7 |
| Xanthan gum | 0.3 |
| Methylcellulose | 0.3 |
| PEG-40 stearate | 3 |
| Polysorbate 80 | 1 |
| Glyceryl monostearate | 0.5 |
| Methylparaben | 0.15 |
| Propylparaben | 0.05 |
| Phenoxyethanol | 1 |
| Metronidazole | 1 |
| Mineral oil | 6 |
| Stearic acid | 1 |
| Isopropyl myristate | 6 |
| Propellant gas | 16 |

The aerosol container filled with the above formulation delivers a metronidazole-based foam which is found to be particularly suitable for application of metronidazole to the skin.

### Example 2

### Preparation of a Formulation F2 Based on C8-C10 Fatty Acid Triglycerides for Delivering a Metronidazole-Based Foam

A metronidazole-based emulsion E2 similar to that of Example 1 was prepared according to the same protocol, the only difference being that the isopropyl myristate used as emollient agent was replaced with a mixture of C8-C10 fatty acid triglycerides (Mygliol).

An emulsion E2 of the oil-in-water type was thus obtained which has the composition given in Table III below, in which the percentages indicated are expressed by mass relative to the total mass of the emulsion.

TABLE III
Composition of the Emulsion E2:

| Constituent | Content (% by mass) |
|---|---|
| Purified water | 75.83% |
| Xanthan gum | 0.36% |
| Methylcellulose | 0.36% |
| PEG-40 stearate | 3.57% |
| Polysorbate 80 | 1.19% |
| Glyceryl monostearate | 0.60% |
| Methylparaben | 0.18% |
| Propylparaben | 0.06% |
| Phenoxyethanol | 1.19% |
| Metronidazole | 1.19% |
| Mineral oil | 7.14% |
| Stearic acid | 1.19% |
| Mygliol | 7.14% |

Packaging:

The emulsion E2 was introduced into an aerosol container into which a butane and propane mixture under pressure was introduced, as in Example 1, as propellant gas.

The formulation F2 obtained has the composition indicated in the following Table IV in which the percentages indicated are expressed by mass relative to the total mass of the formulation.

TABLE IV
Composition of the Formulation F2 in the aerosol container:

| Constituent | Content (% by mass) |
|---|---|
| Purified water | 63.7 |
| Xanthan gum | 0.3 |
| Methylcellulose | 0.3 |
| PEG-40 stearate | 3 |
| Polysorbate 80 | 1 |
| Glyceryl monostearate | 0.5 |
| Methylparaben | 0.15 |
| Propylparaben | 0.05 |
| Phenoxyethanol | 1 |
| Metronidazole | 1 |
| Mineral oil | 6 |
| Stearic acid | 1 |
| Mygliol | 6 |
| Propellant gas | 16 |

The aerosol can filled with the above formulation F2 delivers a metronidazole-based foam which is found to be particularly suitable for application of metronidazole to the skin.

### Example 3

### Preparation of a Formulation F3 Based on C8-C10 Fatty Acid Triglycerides and Propylene Glycol for Delivering a Metronidazole-Based Foam

A metronidazole-based emulsion E3 was prepared according to the protocol of Example 1, with the following differences:

the aqueous phase used additionally comprises propylene glycol, as additional ingredient playing the role of absorption promoter in the final foam composition.

as in Example 2, the isopropyl myristate of the hydrophobic phase was replaced with a mixture of C8-C10 fatty acid triglycerides (Mygliol).

By emulsifying the mixture of the aqueous and hydrophobic phases under the conditions of Example 1, an emulsion E3 of the oil-in-water type was obtained which has the composition given in the following Table V in which the percentages indicated are expressed by mass relative to the total mass of the emulsion.

TABLE V
Composition of the Emulsion E3:

| Constituent | Content (% by mass) |
|---|---|
| Purified water | 72.26% |
| Xanthan gum | 0.36% |
| Methylcellulose | 0.36% |
| PEG-40 stearate | 3.57% |
| Polysorbate 80 | 1.19% |
| Glyceryl monostearate | 0.60% |
| Propylene glycol | 3.57% |
| Methylparaben | 0.18% |
| Propylparaben | 0.06% |
| Phenoxyethanol | 1.19% |
| Metronidazole | 1.19% |
| Mineral oil | 7.14% |
| Stearic acid | 1.19% |
| Mygliol | 7.14% |

Packaging:

The emulsion E3 was introduced into an aerosol container. After closing the container, a butane and propane mixture under pressure was introduced as propellant gas. The formulation F3 obtained in this context has the composition given in Table VI below, in which the percentages indicated are expressed by mass relative to the total mass of the formulation.

TABLE VI
Composition of the Formulation F3 in the aerosol container:

| Constituent | Content (% by mass) |
|---|---|
| Purified water | 60.7 |
| Xanthan gum | 0.3 |
| Methylcellulose | 0.3 |
| PEG-40 stearate | 3 |
| Polysorbate 80 | 1 |
| Glyceryl monostearate | 0.5 |
| Propylene glycol | 3 |
| Methylparaben | 0.15 |
| Propylparaben | 0.05 |
| Phenoxyethanol | 1 |
| Metronidazole | 1 |
| Mineral oil | 6 |
| Stearic acid | 1 |
| Mygliol | 6 |
| Propellant gas | 16 |

The aerosol can filled with the above formulation F3 delivers a metronidazole-based foam which is found to be particularly suitable for application of metronidazole to the skin.

### Example 4

### Physical and Chemical Stability of the Compositions According to the Invention

Two compositions according to the invention were tested for their physical and chemical stability. They are the emulsion E3 of Example 3 (called in the tables below formula E3PG) and Example E3 free of propylene glycol 3 (called in the tables below formula E3 without PG)

Chemical Assay by HPLC:

Metrofoam emulsion E3 without PG

| | |
|---|---|
| RT | 40° C. |

| | Metro- | Metro- | Metro- | Metro- |
|---|---|---|---|---|
| | nidazole | nidazole | nidazole | nidazole |
| | % LC | % Initial | % LC | % Initial |
| T = 0 | 99.9* | NA | NA | NA |
| T = 1 Month | 99.6* | 99.7 | 99.4 | 99.5 |
| T = 3 months | 99.6* | 99.7 | 98.0 | 98.1 |

Metrofoam emulsion E3PG

| | |
|---|---|
| RT | 40° C. |

| | Metro- | Metro- | Metro- | Metro- |
|---|---|---|---|---|
| | nidazole | nidazole | nidazole | nidazole |
| | % LC | % Initial | % LC | % Initial |
| T = 0 | 101.3* | NA | NA | NA |
| T = 1 month | 99.5* | 98.2 | 99.2* | 97.9 |
| T = 3 months | 98.5* | 97.2 | 97.9* | 96.6 |

*Absence of 2-methyl-5-nitroimidazole

Viscosities:

### Viscometer: LVDV II+

### Mobile: Small Volume No. 27

### Speed: 2 rpm

Metrofoam emulsion E3 without PG

| | RT | 40° C. |
|---|---|---|
| | Viscosity (mPa · s) | Viscosity (mPa · s) |
| T = 0 | 5679 | — |
| T = 1 month | 6921 | 6636 |
| T = 3 months | 6165 | 6148 |

Metrofoam emulsion E3PG

| | RT | 40° C. |
|---|---|---|
| | Viscosity (mPa · s) | Viscosity (mPa · s) |
| T = 0 | — | — |
| T = 1 month | 7013 | 6977 |
| T = 3 months | 6589 | 6576 |

### Example 5

### Evaluation of the Cumulative Irritation Potential of Various Prototypes of Foam Vehicle Compositions after Repeated Application Under Occlusion in Healthy Subjects

The compositions tested are:

foam vehicle according to the invention of composition E1 of Example 1,

foam vehicle according to the invention of the above composition E3 without Propylene Glycol (PG),

foam vehicle according to the invention of composition E3 with PG,

Metrolotion® vehicle

Metrogel® vehicle.

Compositions:

| | Content (% m/m) | Content | Content |
|---|---|---|---|
| | MetroFoam | (% m/m) | (% m/m) |
| | emulsion E1 | MetroFoam | MetroFoam |
| | according to | emulsion E3 | emulsion E3 |
| Ingredients | Example 1 | without PG | with PG |
| Methylcellulose | 0.36 | 0.36 | 0.36 |
| Xanthan gum | 0.36 | 0.36 | 0.36 |
| Propylene glycol | — | — | 3.60 |
| PEG-40 stearate | 3.60 | 3.60 | 3.60 |
| Polysorbate 80 | 1.20 | 1.20 | 1.20 |
| Glyceryl monostearate | 0.60 | 0.60 | 0.60 |
| Mineral oil | 7.14 | 7.14 | 7.14 |
| Mygliol | — | 7.14 | 7.14 |
| Isopropyl myristate | 7.14 | — | — |
| Stearic acid | 1.20 | 1.20 | 1.20 |
| Methylparaben | 0.18 | 0.18 | 0.18 |
| Propylparaben | 0.06 | 0.06 | 0.06 |
| Phenoxyethanol | 1.20 | 1.20 | 1.20 |
| Purified water | qs 100 | qs 100 | qs 100 |

| | MetroLotion | | MetroGel |
|---|---|---|---|
| | vehicle | | vehicle |
| Ingredients | (% m/m) | Ingredients | (% m/m) |
| Glycerin | 7.0 | Titriplex III | 0.05 |
| Macrogol 400 | 2.0 | Carbopol 980 NF | 0.65 |
| Carbopol 981 NF | 0.15 | Propylene glycol | 3.0 |
| Brij 721 | 3.0 | Propylparaben | 0.02 |
| Marcol 152 | 6.0 | Methylparaben | 0.08 |
| Arlacel 165FL | 3.0 | Sodium hydroxide | pH 5.5 ± 0.5 |
| | | 10% m/m | |
| Lanette 18 | 2.0 | Purified water | qs 100 |
| Benzyl alcohol | 1.3 | | |
| Potassium sorbate | 0.20 | | |
| Mirasil CM5 | 4.0 | | |
| Sodium hydroxide | pH 5.5 ± 0.5 | | |
| 10% m/m | | | |
| Lactic acid 90% | pH 5.5 ± 0.5 | | |
| Purified water | qs 100 | | |

The parameters of the study are the following:

Study of blind randomized intra-individual comparison in a single center:

Conventional methodology of 21-day tolerance study

Irritation scale used:

0=no erythema

0.5=slightly perceptible erythema

1=mild erythema with or without oedema

2=moderate erythema, oedema with or without papule

3=severe erythema, oedema with or without papule

4=erythema, oedema vesicle or bulla.

Treatment

| | Metroni- | | Metrofoam | | Metrofoam | | Metroni- | | Metrofoam | |
|---|---|---|---|---|---|---|---|---|---|---|
| | dazole | | Vehicle | | Vehicle | | dazole | | Vehicle E1 | |
| | Gel | | containing | | E3 with- | | Lotion | | according to | |
| | Vehicle | | E3 with PG | | out PG | | Vehicle | | Example E1 | |
| | N | % | N | % | N | % | N | % | N | % |
| 0 | 19 | 63.33 | 24 | 80.00 | 20 | 66.67 | 19 | 63.33 | 26 | 86.67 |
| 0.5 | 6 | 20.00 | 5 | 16.67 | 9 | 30.00 | 5 | 16.67 | 2 | 6.67 |
| 1 | 5 | 16.67 | 1 | 3.33 | 1 | 3.33 | 6 | 20.00 | 2 | 6.67 |
| ≧2 | 0 | 0.00 | 0 | 0.00 | 0 | 0.00 | 0 | 0.00 | 0 | 0.00 |

Results:

All the vehicles tested exhibited good tolerance.

The vehicles of the foam compositions according to the invention exhibited greater tolerance than that of the metrolotion and metrogel vehicles.

### Example 6

### Optimization of the Preserving System on the Basis of the Emulsions According to the Invention

The following formulas are prepared and then tested on various bacterial strains in order to evaluate the efficacy of the preserving system.

Summary table for the various preserving systems tested:

| | Content | Content | Content |
|---|---|---|---|
| | (% by mass) of | (% by mass) of | (% by mass) of |
| Constituent | Formula F1 | Formula F2 | Formula F3 |
| Purified water | qs 100 | qs 100 | qs 100 |
| Xanthan gum | 0.36 | 0.36 | 0.36 |
| Methylcellulose | 0.36 | 0.36 | 0.36 |
| PEG-40 stearate | 3.6 | 3.6 | 3.6 |
| Polysorbate 80 | 1.2 | 1.2 | 1.2 |
| Glyceryl | 0.6 | 0.6 | 0.6 |
| monostearate | | | |
| Methylparaben | 0.162 | 0.2 | 0.18 |
| Propylparaben | 0.054 | 0.1 | 0.09 |
| Phenoxyethanol | 1.08 | 1.10 | 0.99 |
| Metronidazole | 1.19 | 1.19 | 1.19 |
| Mineral oil | 7.14 | 7.14 | 7.14 |
| Stearic acid | 1.20 | 1.20 | 1.20 |
| Mygliol | 7.14 | 7.14 | 7.14 |

Details of the microbiology results with:

A: log of the concentration of the microorganisms in the products tested at T0
B: control=log of the concentration of the inoculum
C,D,E,F: results expressed as log reduction relative to the control
NR: not required
IN: increase

REFERENCE SAMPLE: METROFOAM EMULSION 1.19% (w/w)
FORMULA/BATCH NUMBER: F1
ANALYSIS NUMBER: 040702
DATE OF ANALYSIS: 26 Oct. 2004

| | | B: | | | | |
|---|---|---|---|---|---|---|
| Micro- | A: | con- | C: | D: | E: | F: |
| organisms | T = 0 | trol | T = 2 D | T = 7 D | T = 14 D | T = 28 D |
| E. coli (USP) | 6.0 | 5.9 | >3.9 | 5.9 | 5.9 | 5.9 |
| S. aureus | 5.8 | 5.8 | >3.8 | 5.8 | 5.8 | 5.8 |
| P. aeruginosa | 5.9 | 6.1 | >4.1 | 6.1 | 6.1 | 6.1 |
| C. albicans | 6.0 | 5.9 | NR | NR | >3.9 | 5.9 |
| A. niger | 5.8 | 5.8 | NR | NR | 2.7 | 5.8 |

REFERENCE SAMPLE: METROFOAM EMULSION 1.19% (w/w)
FORMULA/BATCH NUMBER: F2

| | | B: | | | | |
|---|---|---|---|---|---|---|
| Micro- | A: | con- | C: | D: | E: | F: |
| organisms | T = 0 | trol | T = 2 D | T = 7 D | T = 14 D | T = 28 D |
| E. coli (USP) | 5.9 | 5.9 | >3.9 | 5.9 | 5.9 | 5.9 |
| S. aureus | 5.9 | 5.8 | >3.8 | 5.8 | 5.8 | 5.8 |
| P. aeruginosa | 5.9 | 6.1 | >4.1 | 6.1 | 6.1 | 6.1 |
| C. albicans | 6.0 | 5.9 | NR | NR | >3.9 | 5.9 |
| A. niger | 5.8 | 5.8 | NR | NR | >3.8 | 5.8 |

REFERENCE SAMPLE: METROFOAM EMULSION 1.19% (w/w)
FORMULA/BATCH NUMBER: F3

| | | B: | | | | |
|---|---|---|---|---|---|---|
| Micro- | A: | con- | C: | D: | E: | F: |
| organisms | T = 0 | trol | T = 2 D | T = 7 D | T = 14 D | T = 28 D |
| E. coli (USP) | 6.1 | 5.9 | >3.9 | 5.9 | 5.9 | 5.9 |
| S. aureus | 5.9 | 5.8 | >3.8 | 5.8 | 5.8 | 5.8 |
| P. aeruginosa | 5.9 | 6.1 | >4.1 | 6.1 | 6.1 | 6.1 |
| C. albicans | 6.0 | 5.9 | NR | NR | >3.9 | 5.9 |
| A. niger | 5.8 | 5.8 | NR | NR | 2.8 | 5.8 |

In conclusion, all of the formulas satisfy the U.S. and European pharmacopoeias.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A stable, metronidazole-based oil-in-water emulsion expandable into a topically applicable foam having a firm, creamy and light consistency, said foam also having improved release-penetration capacity, diminished inhibition of skin breathing and useful for the treatment of dermatological skin conditions/afflictions, comprising (a) water, (b) a gelling agent for the aqueous phase thereof, (c) a dermatologically effective amount of metronidazole, (d) mineral oil, (e) a surfactant, (f) a fatty acid, optionally, (g) a metronidazole absorption promoter and (h) a preservative selected from the group consisting of methylparaben, propylparaben, phenoxyethanol and mixtures thereof.

2. A stable metronidazole-based oil-in-water emulsion expandable into a topically applicable foam useful for the treatment of dermatological conditions/afflictions, comprising by mass relative to the total mass of the emulsion:
(a) 72% to 76% of water;
(b) 0.1% to 5% of a gelling agent for the aqueous phase of the emulsion;
(c) 0.75% to 2% of metronidazole;
(d) 5% to 10% of mineral oil;
(e) 0.5% to 10% of a surfactant;
(f) 0.5% to 5% of a fatty acid;
(g) 0% to 4% of a metronidazole absorption promoter (c); and
(h) 0.1% to 5% of a preservative selected from the group consisting of methylparaben, propylparaben, phenoxyethanol and mixtures thereof.

3. The oil-in-water emulsion as defined by claim 2, in which the preservative (h) comprises a mixture of methylparaben and propylparaben with phenoxyethanol.

4. The oil-in-water emulsion as defined by claim 2, in which the preservative comprises a mixture of:
12 to 15% of methylparaben,
4 to 8% of propylparaben, and
78 to 84% of phenoxyethanol,
the percentages being expressed by weight relative to the total weight of the preservative.

5. The oil-in-water emulsion as defined by claim 2, in which the mineral oil (d) content is greater than or equal to 6% by mass relative to the total mass of the emulsion.

6. The oil-in-water emulsion as defined by claim 2, in which the gelling agent (b) comprises a mixture of a natural thickening gum and a cellulose ester.

7. The oil-in-water emulsion as defined by claim 2, in which the gelling agent (b) is present in an amount of from 0.3 and 2% by mass relative to the total mass of the emulsion.

8. The oil-in-water emulsion as defined by claim 2, in which the surfactant (e) comprises a mixture of polyethylene glycol ester, polyoxyethylenated sorbitan ester and monoglyceride.

9. The oil-in-water emulsion as defined by claim 2, in which the surfactant (e) content is from 3 and 8% by mass relative to the total mass of the emulsion.

10. The oil-in-water emulsion as defined by claim 2, in which the fatty acid (f) comprises stearic acid.

11. The oil-in-water emulsion as defined by claim 2, in which the fatty acid (f) content ranges from 1% to 2% by mass relative to the total mass of the emulsion.

12. The oil-in-water emulsion as defined by claim 2, comprising a propylene glycol absorption promoter.

13. The oil-in-water emulsion as defined by claim 2, additionally comprising at least one ester (i) emollient.

14. The oil-in-water emulsion as defined by claim 2, comprising:
(a) 72% to 76% of water;
(b) a gelling system for the aqueous phase of the emulsion, which comprises:
0.2% to 0.5% of xanthan gum; and
0.2% to 0.5% of methylcellulose;
(c) 0.75% to 2% of metronidazole;
(d) 6% to 8% of mineral oil;
(e) a surfactant comprising a mixture of:
2% to 5% of a polyethylene glycol ester;
0.8% to 2% of a polyoxyethylenated sorbitan ester; and
0.4% to 1% of a monoglyceride;
(f) 1% to 1.5% of stearic acid;
(g) 0% to 4% of propylene glycol;
(h) 0.1% to 2% of a preservative selected from the group consisting of methylparaben, propylparaben, phenoxyethanol and mixtures thereof; and
(i) 0.1% to 10% of an emollient ester.

15. The oil-in-water emulsion as defined by claim 2, comprising:
(a) 72% to 76% of water;
(b) a gelling system for the aqueous phase of the emulsion, which comprises:
0.3% to 0.4% of xanthan gum; and
0.3% to 0.4% of methylcellulose;
(c) 0.75% to 2% of metronidazole;
(d) 6.5% to 8.5% of mineral oil;
(e) a surfactant comprising a mixture of:
3 to 4% of PEG-40 stearate;
1% to 1.5% of polysorbate 80 and
0.5% to 0.8% of glyceryl monostearate;
(f) 1% to 1.5% of stearic acid;
(g) 0% to 4% of propylene glycol;
(h) 0.1% to 2% of a preservative selected from the group consisting of methylparaben, propylparaben, phenoxyethanol and mixtures thereof; and
(i) 5% to 9% of isopropyl myristate or fatty acid triglycerides emollient.

16. The oil-in-water emulsion as defined by claim 2, comprising:
(a) from 72% to 76% of water;
(b) a gelling system for the aqueous phase of the emulsion, which comprises:
from 0.3% to 0.4% of xanthan gum; and
from 0.3% to 0.4% of methylcellulose;
(c) 0.75% to 2% of metronidazole;
(d) 6.5% to 7.5% of mineral oil, and
(e) a surfactant comprising a mixture of:
3% to 4% of PEG-40 stearate;
1% to 1.5% of polysorbate 80 and
0.5% to 0.8% of glyceryl monostearate;
(f) 1% to 1.5% of stearic acid;
(g) 2% to 4% of propylene glycol;
(h) 0.1% to 2% of a preservative selected from the group consisting of methylparaben, propylparaben, phenoxyethanol and mixtures thereof; and
(i) 5% to 9% of isopropyl myristate or fatty acid triglycerides emollient.

17. A method for preparing an oil-in-water emulsion as defined by claim 1, comprising the following successive steps:
preparing an aqueous medium A which comprises water (a), the gelling agent (b), the metronidazole (c), the surfactant (e), the preservative (h) of the paraben type, and optionally the absorption promoter (g);
preparing a hydrophobic medium H which comprises the mineral oil (d) and any other constituent of the fatty phase;
mixing the media A and H thus obtained, and emulsifying the system thus formed;
adding the preservative of the phenoxyethanol type upon completion of the emulsification.

18. A method for preparing an oil-in-water emulsion as defined by claim 1, comprising the following successive steps:
preparing an aqueous medium A which comprises water (a), the gelling agent (b), the metronidazole (c), the surfactant system (e), the propylene glycol (g) and the preservative of the paraben type (h);
preparing a hydrophobic medium H which comprises the mineral oil (d), stearic acid (f) and the ester (i); and
mixing the media A and H thus obtained and emulsifying the system thus formed;
adding the preservative of phenoxyethanol type upon completion of the emulsification.

19. A method for preparing a metronidazole-based foam composition, comprising expanding an oil-in-water emulsion as defined by claim 1 with a gas.

20. The method as defined by claim 19, in which a mixture is obtained by introducing the emulsion into an aerosol can with a propellant gas under pressure, and then releasing the formulation thus obtained.

21. An aerosol can for delivering a foam composition, comprising an emulsion as defined by claim 1, and a propellant gas under pressure.

22. A metronidazole-based foam composition having diminished inhibition of skin breathing, comprising the expanded oil-in-water emulsion as defined by claim 1.

23. A regime or regimen for the treatment of a dermatological skin condition/affliction, comprising topically applying onto the affected skin area of an individual in need of such treatment, the expandable metronidazole-based foam composition as defined by claim 22.

24. The regime or regimen as defined by claim 23, comprising the treatment of rosacea.

25. The regime or regimen as defined by claim 23, comprising the treatment of acne.
